(19) United States
(12) Patent Application Publication
(10) Pub. No.: US 2005/0158805 A1
(43) Pub. Date: Jul. 21, 2005

(54) Toxicity test

(76) Inventors: Wendy Purcell, Bristol (GB); Jinsheng Xu, Bristol (GB)

Correspondence Address: SUGHRUE MION, PLLC, 2100 PENNSYLVANIA AVENUE, N.W., SUITE 800, WASHINGTON, DC, 20037, US

(21) Appl. No.: 10/500,559

(22) PCT Filed: Jan. 14, 2003
(86) PCT No.: PCT/GB03/00097

(30) Foreign Application Priority Data
Jan. 14, 2002 (GB) 0200721.9

(51) Int. Cl. G01N 33/50

(52) U.S. Cl. 435/7.21

(57) ABSTRACT

An in vitro toxicity assay comprising: a) exposing a spheroid sample to a selected concentration of a compound to be assayed; b) incubating the spheroid sample for a suitable period of time; and c) observing if spheroid cell spreading inhibition takes place.

The present invention relates to an in vitro toxicity assay using spheroids.

Spheroids have significant potential to be used as in vitro models to test the toxicity of compounds at various concentrations. The use of spheroids in an in vitro model as a repeatable and reliable indicator of toxicity is a desirable alternative to using live animals.

The inventors have developed a cell spreading inhibition test based on the observed spheroid cell growth, or “spreading” of cells in spheroids when the spheroids are grown on a suitable surface and at static state i.e. without shaking. As shown in FIG. 1, the cells grow out from the surface of the spheroid. It was found that when spheroids are exposed to a toxicant at a certain concentration, cell spreading is inhibited. This inhibition of cell spreading provides an indicator of toxicity.

According to a first aspect of the invention, there is provided an in vitro toxicity assay comprising:
a) exposing a spheroid sample to a selected concentration of a compound to be assayed;
b) incubating the spheroid sample for a suitable period of time; and
c) observing if spheroid cell spreading inhibition takes place.
   At the selected concentration, if no cell spreading, or very limited spreading, of all the spheroids in the sample is observed relative to a control spheroid not exposed to the compound being assayed, this is considered to be a spreading inhibition positive (+) result. However, if there is observable cell spreading from one or more of the spheroids in the sample, this is a cell spreading inhibition negative (−) result.

In the event that partial spreading is observed ie. there is some observable cell spreading in the sample, but not as extensive as in the control, then the assay should be repeated at a higher compound concentration to ensure that a conclusive result can be obtained, ie. a spreading inhibition positive (+) result.

When spheroid cell spreading inhibition occurs, this indicates that, at the selected concentration, the compound has a toxic effect on the cell type from which the spheroid sample is derived.

As used herein, the term “spheroid” means a three dimensional structure, typically substantially spherical in shape, which does not occur in nature and which consists of a re-aggregate of cells—typically containing 10³ or more cells—of a tissue or of an organ or formed from cells lines either alone or in combination.

The term “tissue” as used herein is taken to mean an organised selection of cells having a common function. The term “organ” is taken to mean an organised collection of “tissues” having a common function. The term “cell line” is taken to mean a continuous cell culture derived from cells subject to transformation or otherwise having acquired to ability to divide continuously.

The “tissue” or “organ” need not be completely intact to be used in the present invention since parts of whole tissues or organs (which may be obtained via biopsies) can be disrupted to individual cells/small groups of cells before being re-aggregated to form spheroids used in the present invention.

Cells for use in producing spheroids for use in the present invention may be derived from any suitable tissue source, including infected tissue. For spheroids comprising neuronal cells (e.g. brain spheroids), foetal tissue is preferred. For spheroids comprising other cells, tissue from both embryonic/foetal and non-foetal (e.g. adult sources) can generally be used. Liver cells are particularly useful since they can be used to produce spheroids which retain some of the characteristics of the liver e.g. albumin secretion, urea secretion, glucose secretion and can therefore be used to model in vitro the metabolism of substances in the liver and to explore general cytotoxic and specific hepatotoxic effects. This is useful, for example, in determining whether or not particular substances are likely to be toxic following metabolism by the liver and/or are directly toxic to the liver cells (i.e. hepatotoxins) or interfere with generic cellular functionality.

Spheroids can, in principle, be produced from any desired tissue or organ from any animal by disrupting a sample-of the tissue or organ, preferably to individual cells or to small groups of cells. For example, mechanical disruption such as by gentle trituration through a Pasteur pipette can be employed for retinal and brain tissues. Alternatively, enzymatic digestion methods can be used, for example, for liver cell dissociation.

Preferably, the spheroids of the present invention are derived from mammalian tissue or organs, such as from human, non-human primate, dog, rodent (including rat and Mouse) or porcine tissue or organs. Alternatively, the spheroids of the present invention may be derived from fish tissue or organs.

Cells from cell lines may also be used. These may be initially cultured as a monolayer to generate more cells; trypsinization may be used for cell dissociation of a monolayer cell culture.

The spheroids used in the present invention may comprise two or more different cell types, whereby observation of cell spreading inhibition indicates that all the cell types making up the spheroid exhibit a toxic effect in response to the toxicant being tested.

Spheroids used in the present invention may be freshly prepared or may be obtained by thawing cryopreserved spheroids. Spheroids may be cryogenically prepared using methods such as that described in WO98/35021.

The in vitro assay of the present invention may be carried out using a series of different selected concentrations of the compound to be assayed, to provide an estimate of the threshold concentration at which the compound becomes toxic to the spheroid cell type.

By carrying out the assay of the present invention using the same compound and different spheroid cell types, it may be possible to determine at what concentrations the compound is toxic to one cell type and not to the other.

### BRIEF DESCRIPTION OF THE DRAWINGS

FIGS. 1A to 1C show spheroid cell spreading when spheroids are grown on a surface at static state;

FIGS. 2A to 2C show spheroid spreading in the presence and absence of specific concentrations of galactosamine;

FIGS. 3A to 3C show spheroid spreading in the presence and absence of specific concentrations of propranolol;

FIGS. 4A and 4B show spheroid spreading in the presence of specific concentrations of diclofenac; and

FIGS. 5A and 5B show spheroid spreading the presence of specific concentrations of paracetamol.

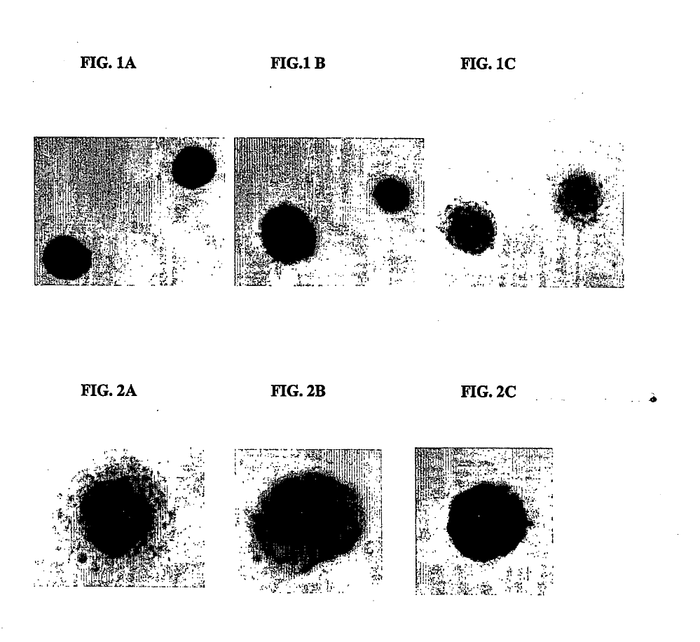

In the following examples, fresh liver or HepG2 spheroids were used. Each test was repeated, to ensure that the same results were obtained and thereby ensure that the spheroid cell spreading inhibition test (SCSIT) is a reliable indicator of toxicity.

Preparation of Liver Spheroids

Liver spheroids were prepared from the liver of male Wistar rats by a two-step collagenase perfusion method described by Seglen P.O. ((1976). Preparation of isolated rat liver cells. Methods Cell. Biol. 13, 29-38) and modified by Lazar, A.; Peshwa, M. V., Wu, F. J., Chi, C. M., Cerra, F. B., and Hu, W. S. (1995). Extended liver-specific functions of procine hepatocyte spheroids entrapped in collagen gel. In Vitro Cell Biol Anim. 31, 340-346). Viability of the isolated liver cells was determined by tyrpan blue dye exclusion i.e. an aliquot of isolated liver cells was mixed with an equal volume of trypan blue dye (1.0% w/v in isotonic saline) and incubated at room temperature for a minimum period of 5 minutes. Only isolated liver cell preparations with viability above 80% were used to prepare liver spheroids. The cell suspension was diluted with culture medium (hepatocyte medium supplemented with 5% FCS, 200 mM L-glutamine, 2 ng/ml insulin, 100 U/ml penicillin and 100 μg/ml streptomycin sulfate) to give a cell density of 5×10⁵ cells/ml. The diluted cell suspension was dispersed into 6-well plates, 3 ml/well. The plates were incubated at 37° C., in a 5% CO₂ incubator on a gyrotatory shaker (New Brunswick) at an initial rotation speed of 85 rpm for the first 24 hr and 77 rpm thereafter. The plates were rotated at this speed for the duration of the study (up to 45 days, but typically 2-10 days). 1.5 ml of old medium was replaced with 2.0 ml fresh medium for each well every other day. Media exchange was carried out over the duration of the study (up to 45 days, but typically 2-10 days).

Spheroids prepared using this protocol have a uniform size typically 170 μm, of which >80% are in the range of 160-180 μm.

Preparation of HepG2 Spheroids

HepG2 cell line (Human Caucasian Hepatocyte Carinomal cells, from ECACC) was cultured as a monolayer in a 75 cm² flask at an initial density of 10² cells/cm² in a culture medium containing MEM (Sigma) supplemented with 10% FBS, 200 nM L-Glutamine, 100 U/ml pencillin and 100 μg/ml streptomycin (GibcoBril). HepG2 cells of confluent flasks were detached by trypsin and pooled together to be counted by trypan blue dye excretion. The cell suspension was diluted with culture medium (MEM supplemented with 5% FBS, 200 nM L-glutamine, 100 U/ml penicillin and 100 μg/ml streptomycin) to give 1×10⁶ cell/ml cell suspension. The cell suspension was plated into 6-well plates, 3 ml/well. The plate was placed on a gyrotatory shaker (New Brunswick) at 83 rpm in a 37° C. CO₂ incubator for the first 24 h, and then the rotation speed reduced to 77 rpm. After 6 DIV culture, spheroids were ready for use.

### EXAMPLE 1

3 to 5 freshly prepared spheroids were transferred into each well of a 24 well plate and exposed to galactosamine at various concentrations as detailed in Tables 1 and 2. Two wells were used for each concentration of galactosamine. The spheroids were incubated in Hepatocyte medium (Sigma) supplemented with 10-15% FCS, 200 nM L-glutamine, 100 U/ml penicillin and 100 μg/ml streptomycim sulfate, and placed in a 5% CO₂ incubator at 37° C. and the effects on spheroid cell spreading inhibition observed after 48 hours exposure of the cells to galactosamine:

TABLE 1
SCSIT results using fresh liver spheroids

| Batch | Control | 4 mM | 10 mM | 16 mM | 20 mM | 40 mM |
|---|---|---|---|---|---|---|
| A | − | − | − | − | + | + |
| B | − | − | − | − | + | + |
| C | − | − | − | − | + | + |

TABLE 2
SCSIT results using fresh HepG2 spheroids

| Batch | Control | 4 mM | 10 mM | 16 mM | 20 mM | 40 mM |
|---|---|---|---|---|---|---|
| A | − | − | − | + | + | + |
| B | − | − | − | + | + | + |
| C | − | − | − | + | + | + |

− indicates that no spheroid cell spreading inhibition was observed;
+ indicates that spheroid cell spreading was inhibited.

FIG. 2A shows a control liver spheroid (with no exposure to galactosamine), 48 hours after cessation of rotation. No inhibition of spheroid cell spreading is observed. FIG. 2B shows that 48 hours after exposure of a liver spheroid to galactosamine at a concentration of 16 mM, no spheroid cell spreading inhibition is observed. However, FIG. 2C shows that 48 hours after exposure of a liver spheroid to galactosamine at a concentration of 40 mM, spheroid cell spreading was inhibited.

From Table 1, it can be seen that galactosamine inhibits liver spheroid cell spreading at concentrations of 20 mM and higher.

From Table 2, it can be seen that galactosamine inhibits HepG2 spheroid cell spreading at concentrations of 16 mM and higher.

### EXAMPLE 2

Freshly prepared spheroids were exposed to propranolol at various concentrations as detailed in Tables 3 and 4, and the effects on spheroid cell spreading inhibition observed:

TABLE 3
SCSIT results using fresh liver spheroids

| Batch | Control | 62.5 μM | 125 μM | 250 μM | 500 μM | 1000 μM |
|---|---|---|---|---|---|---|
| A | − | − | − | + | + | + |
| B | − | − | + | + | + | + |
| C | − | − | + | + | + | + |

TABLE 4
SCSIT results using fresh HepG2 spheroids

| | | | | | | |
|---|---|---|---|---|---|---|
| Batch | Control | 62.5 μM | 125 μM | 250 μM | 500 μM | 1000 μM |

| | | | | | | |
|---|---|---|---|---|---|---|
| A | − | − | + | + | + | + |
| B | − | − | + | + | + | + |
| C | − | − | + | + | + | + |

− indicates that no spheroid cell spreading inhibition was observed;
+ indicates that spheroid cell spreading was inhibited.

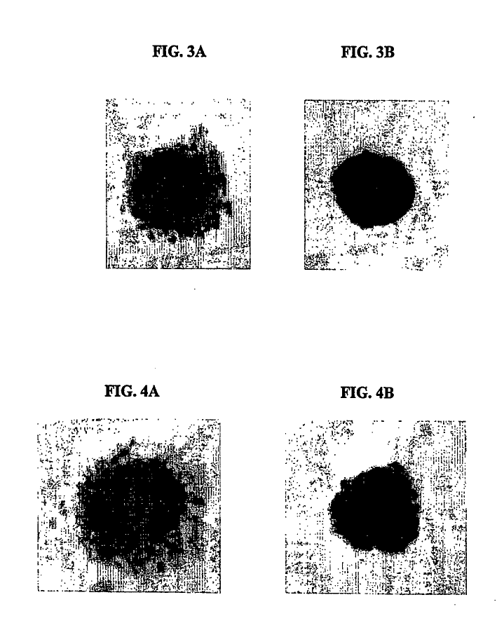

FIG. 3A shows that 48 hours after exposure of a liver spheroid to propranolol at a concentration of 125 μM, no spheroid cell spreading inhibition is observed. However, FIG. 3B shows that 48 hours after exposure of a liver spheroid to propanolol at a concentration of 250 μM, spheroid cell spreading was inhibited.

From Table 3, it can be seen that propanolol inhibits liver spheroid cell spreading at concentrations of 250 μM and higher.

From Table 4, it can be seen that propanolol inhibits HepG2 spheroid cell spreading at concentrations of 125 μm and higher.

### EXAMPLE 3

Freshly prepared spheroids were exposed to diclofenac at various concentrations as detailed in Tables 3 and 4, and the effects on spheroid cell spreading inhibition observed:

TABLE 5
SCSIT results using fresh liver spheroids

| Batch | Control | 0.1 μM | 1 μM | 10 μM | 100 μM | 1000 μM |
|---|---|---|---|---|---|---|
| A | − | − | − | − | − | + |
| B | − | − | − | − | − | + |
| C | − | − | − | − | − | + |

TABLE 6
SCSIT results using fresh HepG2 spheroids

| Batch | Control | 0.1 μM | 1 μM | 10 μM | 100 μM | 1000 μM |
|---|---|---|---|---|---|---|
| A | − | − | − | − | − | + |
| B | − | − | − | − | − | + |
| C | − | − | − | − | − | + |

− indicates that no spheroid cell spreading inhibition was observed;
+ indicates that spheroid cell spreading was inhibited.

FIG. 4A shows that 48 hours after exposure of a liver spheroid to diclofenac at a concentration of 100 μM, no spheroid cell spreading inhibition is observed.

However, FIG. 4B shows that 48 hours after exposure of a liver spheroid to diclofenac at a concentration of 1000 μM, spheroid cell spreading was inhibited.

From Tables 5 and 6, it can be seen that diclofenac inhibits both liver spheroid and HepG2 spheroid cell spreading at concentrations of 1000 μM and higher.

### EXAMPLE 4

Freshly prepared spheroids were exposed to paracetamol at various concentrations as detailed in Tables 3 and 4, and the effects on spheroid cell spreading inhibition observed:

TABLE 7
SCSIT results using fresh liver spheroids

| Batch | Control | 5 μM | 50 μM | 500 μM | 5 mM | 50 mM |
|---|---|---|---|---|---|---|
| A | − | − | − | − | − | + |
| B | − | − | − | − | − | + |
| C | − | − | − | − | − | + |

TABLE 8
SCSIT results using fresh HepG2 spheroids

| Batch | Control | 5 μM | 50 μM | 500 μM | 5 mM | 50 mM |
|---|---|---|---|---|---|---|
| A | − | − | − | − | − | + |
| B | − | − | − | − | − | + |
| C | − | − | − | − | − | + |

− indicates that no spheroid cell spreading inhibition was observed;
+ indicates that spheroid cell spreading was inhibited.

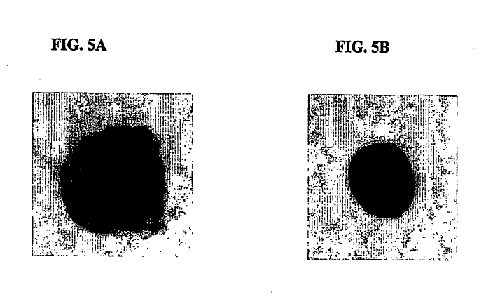

FIG. 5A shows that 48 hours after exposure of a liver spheroid to paracetamol at a concentration of 5 mM, no spheroid cell spreading inhibition is observed.

However, FIG. 5B shows that 48 hours after exposure of a liver spheroid to paracetamol at a concentration of 50 mM, spheroid cell spreading was inhibited.

From Tables 7 and 8, it can be seen that paracetamol inhibits both liver spheroid and HepG2 spheroid cell spreading at concentrations of 50 mM and higher.

Summary

From the examples provided above, it is clear that when spheroids are exposed to a certain concentration of a toxicant, cell spreading is inhibited. This effect was observed with all four selected toxicants using both fresh liver and HepG2 spheroids. Therefore, spheroid cell spreading inhibition is a reliable, repeatable and stable indicator of toxicity.

1. An in vitro toxicity assay comprising:
a) exposing a spheroid sample to a selected concentration of a compound to be assayed;
b) incubating the spheroid sample for a suitable period of time; and
c) observing if spheroid cell spreading inhibition takes place.

2. An in vitro toxicity assay according to claim 1, wherein spheroid cell spreading inhibition indicates that, at the selected concentration, the compound has a toxic effect on the spheroid cell.

3. An in vitro toxicity assay according to claim 1 or claim 2, wherein the spheroid cell is derived from cells selected from the group consisting of neuronal cells, liver cells and retinal cells.

4. An in vitro assay according to claim 1 or claim 2, wherein the spheroid sample is derived from a mammalian cell.

5. An in vitro toxicity assay according to claim 4, wherein the mammalian cell is a human, rodent, or porcine cell.

6. An in vitro toxicity assay according to claim 4, wherein the mammalian cell is a non-human primate or dog cell.

7. An in vitro toxicity assay according to claim 1 or claim 2, wherein the spheroid sample is derived from a fish cell.

8. An in vitro toxicity assay according to claim 1 or claim 2, wherein the spheroid cell is derived from a cultured cell line.

9. An in vitro toxicity assay according to claim 1 or claim 2, wherein the spheroid sample comprises more than one cell type.

10. An in vitro assay according to claim 3, wherein the spheroid sample is derived from a mammalian cell.

11. An in vitro toxicity assay according to claim 10, wherein the mammalian cell is a human, rodent, or porcine cell.

12. An in vitro toxicity assay according to claim 10, wherein the mammalian cell is a non-human primate or dog cell.

13. An in vitro toxicity assay according to claim 3, wherein the spheroid sample is derived from a fish cell.

14. An in vitro toxicity assay according to claim 3, wherein the spheroid cell is derived from a cultured cell line.

15. An in vitro toxicity assay according to claim 4, wherein the spheroid cell is derived from a cultured cell line.

16. An in vitro toxicity assay according to claim 3, wherein the spheroid sample comprises more than one cell type.

17. An in vitro toxicity assay according to claim 4, wherein the spheroid sample comprises more than one cell type.
